(19) United States
(12) Patent Application Publication
(10) Pub. No.: US 2016/0279142 A1
(43) Pub. Date: Sep. 29, 2016

(54) COMBINATIONS OF A PHOSPHOINOSITIDE 3-KINASE INHIBITOR COMPOUND AND A CDK4/6 INHIBITOR COMPOUND FOR THE TREATMENT OF CANCER

(71) Applicant: Genentech, Inc., South San Francisco, CA (US)

(72) Inventors: Lori Friedman, San Carlos, CA (US); Michelle Nannini, Belmont, CA (US); Deepak Sampath, San Francisco, CA (US); Jeffrey Wallin, Berkeley, CA (US)

(73) Assignee: Genentech, Inc., South San Francisco, CA (US)

(21) Appl. No.: 15/080,019

(22) Filed: Mar. 24, 2016

Related U.S. Application Data
(60) Provisional application No. 62/138,556, filed on Mar. 26, 2015.

(51) Int. Cl. A61K 31/553; C12Q 1/68; A61K 31/519

(52) U.S. Cl. CPC A61K 31/553; A61K 31/519; C12Q 1/6886; C12Q 2600/156; C12Q 2600/106; C12Q 2600/158

(57) ABSTRACT

Methods and compositions are provided for treating cancer in patients with a therapeutic combination comprising a therapeutically effective amounts of taselisib and palbociclib, or stereoisomers, geometric isomers, tautomers, or pharmaceutically acceptable salts thereof.

### CROSS REFERENCE TO RELATED APPLICATIONS

This non-provisional application filed under 37 CFR §1.53(b), claims the benefit under 35 USC §119(e) of U.S. Provisional Application Ser. No. 62/138,556 filed on 26 Mar. 2015, which is incorporated by reference in entirety.

### FIELD OF THE INVENTION

The invention relates generally to pharmaceutical combinations of compounds with activity against hyperproliferative disorders such as cancer. The invention also relates to methods of using the compounds for in vitro, in situ, and in vivo diagnosis or treatment of mammalian cells, or associated pathological conditions.

### BACKGROUND OF THE INVENTION

Combinations of anti-cancer pharmaceutical therapeutics administered simultaneously or sequentially in a dosing regimen are now common in cancer treatment. Successful combination therapy provides improved and even synergistic effect over mono-therapy, i.e. pharmaceutical treatment limited to one drug (Ouchi et al (2006) Cancer Chemother. Pharmacol. 57:693-702; Higgins et al (2004) Anti-Cancer Drugs 15:503-512). Preclinical research has been the basis for prediction of clinical stage synergy of anti-cancer pharmaceutical therapeutic combinations such as capecitabine and taxanes for the treatment of breast cancer (Sawada et al (1998) Clin. Cancer Res. 4:1013-1019). Certain doses and schedules of combination therapy can improve safety without compromising efficacy (O' Shaughnessy et al (2006) Clin. Breast Cancer April 7(1):42-50). Synergistic effects in vitro have been correlated with clinical stage synergy (Steinbach et al (2003) Clin. Inf. Dis. October 1:37 Suppl 3:S188-224).

Upregulation of the phosphoinositide-3 kinase (PI3K)/Akt signaling pathway is a common feature in most cancers (Yuan and Cantley (2008) Oncogene 27:5497-510). Genetic deviations in the pathway have been detected in many human cancers (Osaka et al (2004) Apoptosis 9:667-76) and act primarily to stimulate cell proliferation, migration and survival. Activation of the pathway occurs following activating point mutations or amplifications of the PIK3CA gene encoding the p110a PI3K isoforms (Hennessy et al (2005) Nat. Rev. Drug Discov. 4:988-1004). Genetic deletion or loss of function mutations within the tumor suppressor PTEN, a phosphatase with opposing function to PI3K, also increases PI3K pathway signaling (Zhang and Yu (2010) Clin. Cancer Res. 16:4325-30. These aberrations lead to increased downstream signaling through kinases such as Akt and mTOR and increased activity of the PI3K pathway has been proposed as a hallmark of resistance to cancer treatment (Opel et al (2007) Cancer Res. 67:735-45; Razis et al (2011) Breast Cancer Res. Treat. 128:447-56).

Phosphatidylinositol 3-Kinase (PI3K) is a major signaling node for key survival and growth signals for lymphomas and is opposed by the activity of the phosphatase PTEN. The PI3K pathway is dysregulated in aggressive forms of lymphoma (Abubaker (2007) Leukemia 21:2368-2370). Eight percent of DLBCL (diffuse large B-cell lymphoma) cancers have PI3CA (phosphatidylinositol-3 kinase catalytic subunit alpha) missense mutations and 37% are PTEN negative by immunohistochemistry test.

Phosphatidylinositol is one of a number of phospholipids found in cell membranes, and which participate in intracellular signal transduction. Cell signaling via 3′-phosphorylated phosphoinositides has been implicated in a variety of cellular processes, e.g., malignant transformation, growth factor signaling, inflammation, and immunity (Rameh et al (1999) J. Biol Chem. 274:8347-8350). The enzyme responsible for generating these phosphorylated signaling products, phosphatidylinositol 3-kinase (also referred to as PI 3-kinase or PI3K), was originally identified as an activity associated with viral oncoproteins and growth factor receptor tyrosine kinases that phosphorylate phosphatidylinositol (PI) and its phosphorylated derivatives at the 3′-hydroxyl of the inositol ring (Panayotou et al (1992) Trends Cell Biol 2:358-60). Phosphoinositide 3-kinases (PI3K) are lipid kinases that phosphorylate lipids at the 3-hydroxyl residue of an inositol ring (Whitman et al (1988) Nature, 332:664). The 3-phosphorylated phospholipids (PIP3s) generated by PI3-kinases act as second messengers recruiting kinases with lipid binding domains (including plekstrin homology (PH) regions), such as Akt and PDK1, phosphoinositide-dependent kinase-1 (Vivanco et al (2002) Nature Rev. Cancer 2:489; Phillips et al (1998) Cancer 83:41).

The PI3 kinase family comprises at least 15 different enzymes sub-classified by structural homology and are divided into 3 classes based on sequence homology and the product formed by enzyme catalysis. The class I PI3 kinases are composed of 2 subunits: a 110 kd catalytic subunit and an 85 kd regulatory subunit. The regulatory subunits contain SH2 domains and bind to tyrosine residues phosphorylated by growth factor receptors with a tyrosine kinase activity or oncogene products, thereby inducing the PI3K activity of the p110 catalytic subunit which phosphorylates its lipid substrate. Class I PI3 kinases are involved in important signal transduction events downstream of cytokines, integrins, growth factors and immunoreceptors, which suggests that control of this pathway may lead to important therapeutic effects such as modulating cell proliferation and carcinogenesis. Class I PI3Ks can phosphorylate phosphatidylinositol (PI), phosphatidylinositol-4-phosphate, and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate. Class III PI3Ks can only phosphorylate PI. A key PI3-kinase isoform in cancer is the Class I PI3-kinase, p110a as indicated by recurrent oncogenic mutations in p110α (Samuels et al (2004) Science 304:554; U.S. Pat. No. 5,824,492; U.S. Pat. No. 5,846,824; U.S. Pat. No. 6,274,327). Other isoforms may be important in cancer and are also implicated in cardiovascular and immune-inflammatory disease (Workman P (2004) Biochem Soc Trans 32:393-396; Patel et al (2004) Proc. Am. Assoc. of Cancer Res. (Abstract LB-247) 95th Annual Meeting, March 27-31, Orlando, Fla., USA; Ahmadi K and Waterfield M D (2004) “Phosphoinositide 3-Kinase: Function and Mechanisms” Encyclopedia of Biological Chemistry (Lennarz W J, Lane M D eds) Elsevier/Academic Press), Oncogenic mutations of p110 alpha have been found at a significant frequency in colon, breast, brain, liver, ovarian, gastric, lung, and head and neck solid tumors. About 35-40% of hormone receptor positive (HR+) breast cancer tumors harbor a PIK3CA mutation. PTEN abnormalities are found in glioblastoma, melanoma, prostate, endometrial, ovarian, breast, lung, head and neck, hepatocellular, and thyroid cancers.

PI3 kinase (PI3K) is a heterodimer consisting of p85 and p110 subunits (Otsu et al (1991) Cell 65:91-104; Hiles et al (1992) Cell 70:419-29). Four distinct Class I PI3Ks have been identified, designated PI3K α (alpha), β (beta), δ (delta), and ω (gamma), each consisting of a distinct 110 kDa catalytic subunit and a regulatory subunit. Three of the catalytic subunits, i.e., p110 alpha, p110 beta and p110 delta, each interact with the same regulatory subunit, p85; whereas p110 gamma interacts with a distinct regulatory subunit, p101. The patterns of expression of each of these PI3Ks in human cells and tissues are distinct. In each of the PI3K alpha, beta, and delta subtypes, the p85 subunit acts to localize PI3 kinase to the plasma membrane by the interaction of its SH2 domain with phosphorylated tyrosine residues (present in an appropriate sequence context) in target proteins (Rameh et al (1995) Cell, 83:821-30; Volinia et al (1992) Oncogene, 7:789-93).

Measuring expression levels of biomarkers (e.g., secreted proteins in plasma) can be an effective means to identify patients and patient populations that will respond to specific therapies including, e.g., treatment with chemotherapeutic agents. There is a need for more effective means for determining which patients with hyperproliferative disorders such as cancer will respond to which treatment with chemotherapeutic agents, and for incorporating such determinations into more effective treatment regimens for patients, whether the chemotherapeutic agents are used as single agents or combined with other agents.

The PI3 kinase/Akt/PTEN pathway is an attractive target for cancer drug development since such agents would be expected to inhibit cellular proliferation, to repress signals from stromal cells that provide for survival and chemoresistance of cancer cells, to reverse the repression of apoptosis and surmount intrinsic resistance of cancer cells to cytotoxic agents. PI3 kinase inhibitors have been reported (Yaguchi et al (2006) Jour. of the Nat. Cancer Inst. 98(8):545-556; U.S. Pat. No. 7,173,029; U.S. Pat. No. 7,037,915; U.S. Pat. No. 6,608,056; U.S. Pat. No. 6,608,053; U.S. Pat. No. 6,838,457; U.S. Pat. No. 6,770,641; U.S. Pat. No. 6,653,320; U.S. Pat. No. 6,403,588; U.S. Pat. No. 7,750,002; WO 2006/046035; U.S. Pat. No. 7,872,003; WO 2007/042806; WO 2007/042810; WO 2004/017950; US 2004/092561; WO 2004/007491; WO 2004/006916; WO 2003/037886; US 2003/149074; WO 2003/035618; WO 2003/034997; US 2003/158212; EP 1417976; US 2004/053946; JP 2001247477; JP 08175990; JP 08176070).

Certain thienopyrimidine compounds have p110 alpha binding, PI3 kinase inhibitory activity, and inhibit the growth of cancer cells (Wallin et al (2011) Mol. Can. Ther. 10(12):2426-2436; Sutherlin et al (2011) Jour. Med. Chem. 54:7579-7587; US 2008/0207611; U.S. Pat. No. 7,846,929; U.S. Pat. No. 7,781,433; US 2008/0076758; U.S. Pat. No. 7,888,352; US 2008/0269210. Pictilisib (pictrelisib, GDC-0941, RG-7321, Genentech Inc., CAS Reg. No. 957054-30-7) is a potent multitargeted class I (pan) inhibitor of PI3K isoforms and in phase II clinical trials for the treatment of advanced solid tumors. Pictilisib is named as 4-(2-(1H-indazol-4-yl)-6-((4-(methylsulfonyl)piperazin-1-yl)methyl)thieno[3,2-d]pyrimidin-4-yl)morpholine (U.S. Pat. No. 7,781,433; U.S. Pat. No. 7,750,002; Folkes et al (2008) Jour. of Med. Chem. 51(18):5522-5532; U.S. Pat. No. 7,781,433; Belvin et al, American Association for Cancer Research Annual Meeting 2008, 99th:April 15, Abstract 4004; Folkes et al, American Association for Cancer Research Annual Meeting 2008, 99th:April 14, Abstract LB-146; Friedman et al, American Association for Cancer Research Annual Meeting 2008, 99th:April 14, Abstract LB-110). Pictilisib shows synergistic activity in vitro and in vivo in combination with certain chemotherapeutic agents against solid tumor cell lines (U.S. Pat. No. 8,247,397).

Taselisib (GDC-0032, Roche RG7604, CAS Reg. No. 1282512-48-4, Genentech Inc.), named as 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide, has potent PI3K activity (WO 2011/036280; U.S. Pat. No. 8,242,104; U.S. Pat. No. 8,343,955) and is being studied in patients with locally advanced or metastatic solid tumors.

Loss of cell cycle control is a hallmark of cancer. Cyclin-dependent kinases CDK 4/6 are highly active in numerous cancers, leading to loss of proliferative control (Shapiro G I (2006) J Clin Oncol.; 24(11):1770-1783; Weinberg R A. (2013) The Biology of Cancer. New York, N.Y. Garland Science). Cell cycle regulators CDK 4/6 trigger cellular progression from growth phase (G1) into S phases associated with DNA replication (Hirama T and H. Phillip Koeffler. (1995) Blood.; 86:841-854; Fry D et al (2004) Molecular Cancer Therapeutics.; 3:1427-1437). CDK 4/6, whose increased activity is frequent in estrogen receptor-positive (ER+) breast cancer (BC), are key downstream targets of ER signaling in ER+BC (Finn R S et al. (2009) Breast Cancer Res.; 11(5):R77; Lamb R, et al (2013) Cell Cycle; 12(15):2384-2394). Preclinical data suggests that dual inhibition of CDK 4/6 and ER signaling stops growth of ER+BC cell lines in the G1 phase.

Palbociclib (PD-0332991, IBRANCE®, Pfizer, Inc.) is an approved drug (Pfizer Inc.) for the treatment of advanced (metastatic) breast cancer and a selective inhibitor of the cyclin-dependent kinases CDK4 and CDK6 (Finn et al (2009) Breast cancer research: BCR 11 (5):R77; Rocca et al (2014) Expert Opin Pharmacother 15 (3):407-20; U.S. Pat. No. 7,863,278; U.S. Pat. No. 7,208,489; U.S. Pat. No. 7,456,168). Palbociclib can be prepared and characterized as described in U.S. Pat. No. 7,345,171. The combination of palbociclib and letrozole (FEMARA®, Novartis Inc.) compared with letrozole alone showed significant and clinically meaningful improvement in progression-free survival (PFS) of post-menopausal women with estrogen receptor positive (ER+), human epidermal growth factor receptor 2 negative (HER2−) locally advanced or newly diagnosed metastatic breast cancer (Pfizer Inc., Press Release, 3 Feb. 2014).

### SUMMARY OF THE INVENTION

It has been determined that additive or synergistic effects in inhibiting the growth of cancer cells in vitro and in vivo can be achieved by administering taselisib (GDC-0032, Genentech Inc.) in combination with palbociclib (PD-0332991, IBRANCE®, Pfizer, Inc.), or pharmaceutically acceptable salts thereof. The combinations and methods may be useful in the treatment of hyperproliferative disorders such as cancer.

Taselisib and palbociclib have the structures:

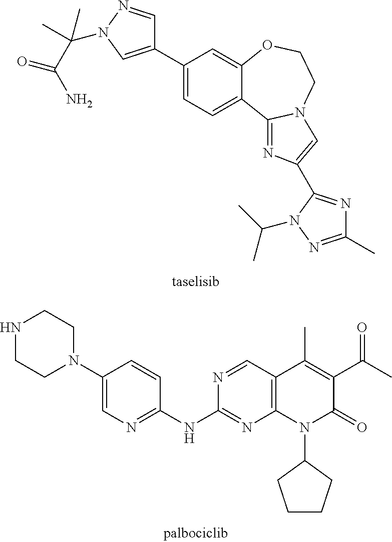 [SMILES: Cc1nc(-c2cn3c(n2)-c2ccc(-c4cnn(C(C)(C)C(N)=O)c4)cc2OCC3)n(C(C)C)n1 ; CC(=O)c1c(C)c2cnc(Nc3ccc(N4CCNCC4)cn3)nc2n(C2CCCC2)c1=O]

or stereoisomers, geometric isomers, tautomers, or pharmaceutically acceptable salts thereof.

### BRIEF DESCRIPTION OF THE DRAWINGS

FIGS. 1a-c show plots of the effects of GDC-0032 (taselisib), palbociclib, and the combination of GDC-0032+palbociclib on a MCF7 breast cancer cell line engineered to expressed aromatase (MCF7×2.3.ARO); Parental (FIG. 1a), letrozole resistant, Letrozole-R1 (FIG. 1b), and double resistant, Let-R1, GDC-0032-R (FIG. 1c). An in vitro assay (CellTiter-Glo® Luminescent Cell Viability Assay, Promega Corp.) measured viable cells in CTG (CellTiter-Glo®) units. Starting doses for GDC-0032 were 80 nM for the parental and letrozole-R1 lines and 10 μM for GDC-0032. Palbociclib starting doses were 10 μM for all three lines. The letrozole/GDC-0032 double resistant cell line is sensitive to the GDC-0032+palbociclib combination.

FIG. 2 shows pathway signaling effects by western blot autoradiograms of gel electrophoresis of cell lysates collected after 24 hours of exposure to no drug, GDC-0032, palbociclib, and the combination of GDC-0032+palbociclib in the Parental, letrozole-resistant, Letrozole-R1, and double-resistant, Let-R1, GDC-0032-R cell lines. Cells were treated for 24 hours with 20 nM GDC-0032 (Parental and Letrozole-R1) or 2.5 μM (Let-R1.GDC-0032-R) and/or 2.5 μM palbociclib.

FIG. 3 shows a plot of in vitro cell proliferation data with MCF7×2.3.ARO breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. An in vitro assay (CellTiter-Glo® Luminescent Cell Viability Assay, Promega Corp.) measured viable cells in CTG (CellTiter-Glo®) units.

FIG. 4 shows a plot of in vitro cell proliferation data with MCF7×2.3.ARO.LetR letrozole-resistant breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. An in vitro assay (CellTiter-Glo® Luminescent Cell Viability Assay, Promega Corp.) measured viable cells in CTG (CellTiter-Glo®) units.

FIG. 5 shows a plot of in vitro cell proliferation data with MCF7×2.3.CMV.ARO breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. An in vitro assay (CellTiter-Glo® Luminescent Cell Viability Assay, Promega Corp.) measured viable cells in CTG (CellTiter-Glo®) units.

FIG. 6 shows a plot of in vitro cell proliferation data with MCF7×2.3.CMV.ARO.LetR letrozole-resistant breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. An in vitro assay (CellTiter-Glo® Luminescent Cell Viability Assay, Promega Corp.) measured viable cells in CTG (CellTiter-Glo®) units.

FIG. 7 shows a plot of in vivo tumor volume change over 22 days in cohorts of immunocompromised mice bearing MCF-7 breast cancer xenografts, dosed daily for 21 days by PO (oral) administration with: vehicle, GDC-0941 (pictilisib) at 75 mg/kg, GDC-0032 at 5 mg/kg, palbociclib at 50 mg/kg, the combination of GDC-0941 at 75 mg/kg+palbociclib at 50 mg/kg, and the combination of GDC-0032 at 5 mg/kg+palbociclib at 50 mg/kg.

FIG. 8 shows a plot of in vivo tumor volume change over 16 days in cohorts of immunocompromised mice bearing MDA-MB-453 xenografts that is hormone receptor negative (HR neg), HER2 positive (HER2+), and harbors a PIK3CA mutation (H1047R), dosed daily for 21 days by PO (oral) administration with: vehicle, GDC-0032 at 5 mg/kg, palbociclib at 50 mg/kg, and the combination of GDC-0032 at 5 mg/kg+palbociclib at 50 mg/kg.

FIGS. 9a-d show ratios of protein levels of mice treated with vehicle, GDC-0032 at 5 mg/kg, palbociclib at 50 mg/kg, and the combination of GDC-0032 at 5 mg/kg+palbociclib at 50 mg/kg, measured at 1 hr and 4 hr. FIG. 9a shows the ratio of phosphoAkt (pAkt) to total Akt (tAkt). FIG. 9b shows the ratio of phospho PRAS40 (pPRAS40) to total PRAS40 (tPRAS40). FIG. 9c shows the ratio of phospho S6RP (pS6RP) to total S6RP (tS6RP). FIG. 9d shows the ratio of phosphor Rb (pRb) to total Rb (tRb).

FIG. 9e shows the concentration of cleaved PARP [ng/mL] of mice treated with vehicle, GDC-0032 at 5 mg/kg, palbociclib at 50 mg/kg, and the combination of GDC-0032 at 5 mg/kg+palbociclib at 50 mg/kg, measured at 1 hr and 4 hr.

FIGS. 10a and 10b shows pathway signaling effects by western blot autoradiograms of gel electrophoresis of cell lysates collected after 1 hour (FIG. 10a) and 4 hours (FIG. 10b) of exposure to no drug (Vehicle), GDC-0032 at 5 mg/kg, palbociclib at 50 mg/kg, and the combination of GDC-0032+palbociclib in the MDA-MB-453 xenograft. Levels of CDK2, CDK4, cyclin D1, cyclin E2, p21, and Actin were visualized.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### DEFINITIONS

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “detection” includes any means of detecting, including direct and indirect detection.

The term “diagnosis” is used herein to refer to the identification or classification of a molecular or pathological state, disease or condition. For example, “diagnosis” may refer to identification of a particular type of cancer, e.g., a lung cancer. “Diagnosis” may also refer to the classification of a particular type of cancer, e.g., by histology (e.g., a non small cell lung carcinoma), by molecular features (e.g., a lung cancer characterized by nucleotide and/or amino acid variation(s) in a particular gene or protein), or both.

The term “prognosis” is used herein to refer to the prediction of the likelihood of cancer-attributable death or progression, including, for example, recurrence, metastatic spread, and drug resistance, of a neoplastic disease, such as cancer.

The term “prediction” (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity 5 of treatment.

“Patient response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

A “biomarker” is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity, and enables optimization of dose and administration schedule. A “biomarker mutation” is a mutation in the wild type form of a protein biomarker.

“Change” or “modulation” of the status of a biomarker, including a PIK3CA mutation or set of PIK3CA mutations, as it occurs in vitro or in vivo is detected by analysis of a biological sample using one or more methods commonly employed in establishing pharmacodynamics (PD), including: (1) sequencing the genomic DNA or reverse-transcribed PCR products of the biological sample, whereby one or more mutations are detected; (2) evaluating gene expression levels by quantitation of message level or assessment of copy number; and (3) analysis of proteins by immunohistochemistry, immunocytochemistry, ELISA, or mass spectrometry whereby degradation, stabilization, or post-translational modifications of the proteins such as phosphorylation or ubiquitination is detected.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Gastric cancer, as used herein, includes stomach cancer, which can develop in any part of the stomach and may spread throughout the stomach and to other organs; particularly the esophagus, lungs, lymph nodes, and the liver.

The term “hematopoietic malignancy” refers to a cancer or hyperproliferative disorder generated during hematopoiesis involving cells such as leukocytes, lymphocytes, natural killer cells, plasma cells, and myeloid cells such as neutrophils and monocytes. Hematopoietic malignancies include non-Hodgkin's lymphoma, diffuse large hematopoietic lymphoma, follicular lymphoma, mantle cell lymphoma, chronic lymphocytic leukemia, multiple myeloma, acute myelogenous leukemia, and myeloid cell leukemia. Lymphocytic leukemia (or “lymphoblastic”) includes Acute lymphoblastic leukemia (ALL) and Chronic lymphocytic leukemia (CLL). Myelogenous leukemia (also “myeloid” or “nonlymphocytic”) includes Acute myelogenous (or Myeloblastic) leukemia (AML) and Chronic myelogenous leukemia (CML).

A “chemotherapeutic agent” is a biological (large molecule) or chemical (small molecule) compound useful in the treatment of cancer, regardless of mechanism of action.

The term “mammal” includes, but is not limited to, humans, mice, rats, guinea pigs, monkeys, dogs, cats, horses, cows, pigs and sheep.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton Pa.; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the mammal being treated therewith.

The term “synergistic” as used herein refers to a therapeutic combination which is more effective than the additive effects of the two or more single agents. A determination of a synergistic interaction between a compound of GDC-0032 or a pharmaceutically acceptable salt thereof and one or more chemotherapeutic agent may be based on the results obtained from the assays described herein. The results of these assays can be analyzed using the Chou and Talalay combination method and Dose-Effect Analysis with CalcuSyn software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents, such as described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 and 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or in separate pills or tablets. In general, during alternation therapy, an effective dosage of each active ingredient is administered sequentially, i.e., serially, whereas in combination therapy, effective dosages of two or more active ingredients are administered together. Combination effects were evaluated using both the BLISS independence model and the highest single agent (HSA) model (Lehár et al. 2007, Molecular Systems Biology 3:80). BLISS scores quantify degree of potentiation from single agents and a BLISS score>0 suggests greater than simple additivity. An HSA score>0 suggests a combination effect greater than the maximum of the single agent responses at corresponding concentrations.

“ELISA” (Enzyme-linked immunosorbent assay) is a popular format of a “wet-lab” type analytic biochemistry assay that uses one sub-type of heterogeneous, solid-phase enzyme immunoassay (EIA) to detect the presence of a substance in a liquid sample or wet sample (Engvall E, Perlman P (1971). “Enzyme-linked immunosorbent assay (ELISA). Quantitative assay of immunoglobulin G”. Immunochemistry 8 (9): 871-4; Van Weemen B K, Schuurs A H (1971). “Immunoassay using antigen-enzyme conjugates”. FEBS Letters 15 (3): 232-236). ELISA can perform other forms of ligand binding assays instead of strictly “immuno” assays, though the name carried the original “immuno” because of the common use and history of development of this method. The technique essentially requires any ligating reagent that can be immobilized on the solid phase along with a detection reagent that will bind specifically and use an enzyme to generate a signal that can be properly quantified. In between the washes only the ligand and its specific binding counterparts remain specifically bound or “immunosorbed” by antigen-antibody interactions to the solid phase, while the nonspecific or unbound components are washed away. Unlike other spectrophotometric wet lab assay formats where the same reaction well (e.g. a cuvette) can be reused after washing, the ELISA plates have the reaction products immunosorbed on the solid phase which is part of the plate and thus are not easily reusable. Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody that is linked to an enzyme through bioconjugation. Between each step, the plate is typically washed with a mild detergent solution to remove any proteins or antibodies that are not specifically bound. After the final wash step, the plate is developed by adding an enzymatic substrate to produce a visible signal, which indicates the quantity of antigen in the sample.

“Immunohistochemistry” (IHC) refers to the process of detecting antigens (e.g., proteins) in cells of a tissue section by exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). IHC is also widely used to understand the distribution and localization of biomarkers and differentially expressed proteins in different parts of a biological tissue. Visualizing an antibody-antigen interaction can be accomplished in a number of ways. In the most common instance, an antibody is conjugated to an enzyme, such as peroxidase, that can catalyze a color-producing reaction (see immunoperoxidase staining). Alternatively, the antibody can also be tagged to a fluorophore, such as fluorescein or rhodamine (see immunofluorescence).

“Immunocytochemistry” (ICC) is a common laboratory technique that uses antibodies that target specific peptides or protein antigens in the cell via specific epitopes. These bound antibodies can then be detected using several different methods. ICC can evaluate whether or not cells in a particular sample express the antigen in question. In cases where an immunopositive signal is found, ICC also determines which sub-cellular compartments are expressing the antigen.

### Taselisib

The compound known as taselisib, GDC-0032, and Roche RG7604, Genentech Inc., CAS Reg. No. 1282512-48-4), has an IUPAC name: 2-(4-(2-(1-isopropyl-3-methyl-1H-1,2,4-triazol-5-yl)-5,6-dihydrobenzo[f]imidazo[1,2-d][1,4]oxazepin-9-yl)-1H-pyrazol-1-yl)-2-methylpropanamide, and the structure:

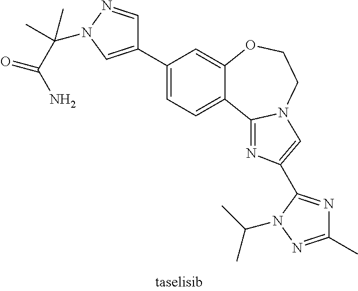 [SMILES: Cc1nc(-c2cn3c(n2)-c2ccc(-c4cnn(C(C)(C)C(N)=O)c4)cc2OCC3)n(C(C)C)n1]

including stereoisomers, geometric isomers, tautomers, and pharmaceutically acceptable salts thereof.

Taselesib can be prepared and characterized as described in WO 2011/036280, U.S. Pat. No. 8,242,104, and U.S. Pat. No. 8,343,955.

### Palbociclib

The compound known as palbociclib (PD-0332991, IBRANCE®, Pfizer, Inc., CAS Reg. No. 571190-30-2) has an IUPAC name: 6-acetyl-8-cyclopentyl-5-methyl-2-(5-(piperazin-1-yl)pyridin-2-ylamino)pyrido[2,3-d]pyrimidin-7(8H)-one, and the structure

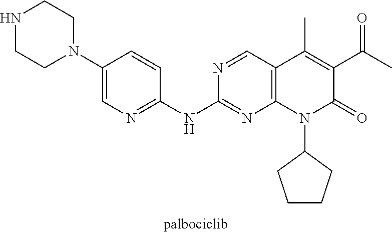 [SMILES: CC(=O)c1c(C)c2cnc(Nc3ccc(N4CCNCC4)cn3)nc2n(C2CCCC2)c1=O]

IBRANCE® is approved for the treatment of breast cancer. Palbociclib is a selective inhibitor of the cyclin-dependent kinases CDK4 and CDK6 (Finn et al (2009) Breast cancer research: BCR 11 (5):R77; Rocca et al (2014) Expert Opin Pharmacother 15 (3):407-20; U.S. Pat. No. 6,936,612; U.S. Pat. No. 7,863,278; U.S. Pat. No. 7,208,489; U.S. Pat. No. 7,456,168). Palbociclib can be prepared and characterized as described in U.S. Pat. No. 7,345,171.

### Taselisib and Palbociclib Combination In Vitro Activity

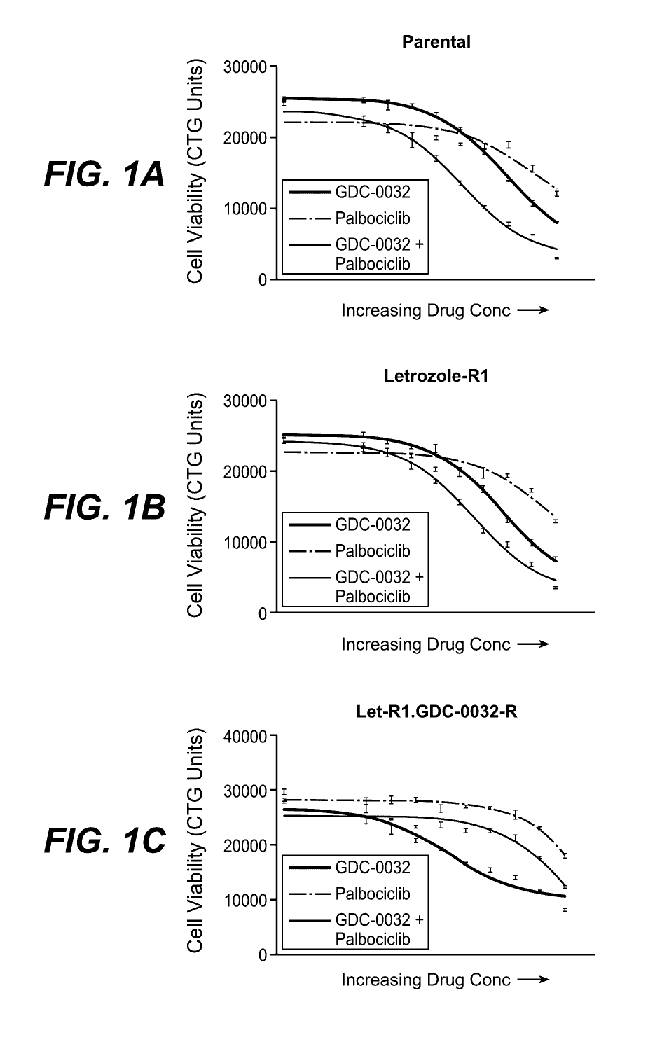

The therapeutic combination of taselisib and palbociclib was tested in parental and resistant cell line models (FIGS. 1a-c). Relative to single agent treatments, decreased viability was observed with the taselisib and palbociclib combination in each cell line model.

Aromatase-expressing MCF7 breast cancer cells (MCF7.ARO) convert androstenedione to estrogen in culture. While most cancer cell lines don't express aromatase, MCF7.ARO can be used as a model to study aromatase inhibitors in combination with PI3K inhibitors and other therapies. FIGS. 1a, 1b and 1c show the single agent (taselisib and palbociclib) and combination effects in MCF7.ARO cells. Single agent in vitro cellular proliferation data in MCF7.ARO parental (FIG. 1a) and letrozole-resistant MCF7 LetR (FIG. 1b) cell lines was collected with taselisib and palbociclib. LetR cells are more resistant to palbociclib and similarly sensitive to taselisib.

Dual resistant cells are still sensitive to taselisib in combination with CDK4/6 inhibition by palbociclib. Taselisib combines well with palbociclib in double-resistant MCF7-ARO cells (FIG. 1c). The effect on viability of taselisib and palbociclib as single agents is shown, respectively. The combination effect of the two drugs is indicated. Starting doses for taselisib were 80 nM for the parental and letrozole-R1 lines and 10 μM for taselisib. Palbociclib starting doses were 10 μM for all three lines (FIGS. 1a-c). Immunoblots from samples treated for 24 hours with 20 nM taselisib (Parental and Letrozole-R1) or 2.5 (Let-R1.taselisib-R). (D) Increased growth arrest is observed with combined PI3K and CDK4/6 inhibition. Immunoblots from samples treated for 24 hours with 20 nM taselisib (Parental and Letrozole-R1) or 2.5 μM (Let-R1. taselisib-R) and/or 2.5 μM palbociclib. Dotted lines for all viability data are indicative of CTG counts at the beginning of drug treatment. Error bars indicate standard deviation around the mean.

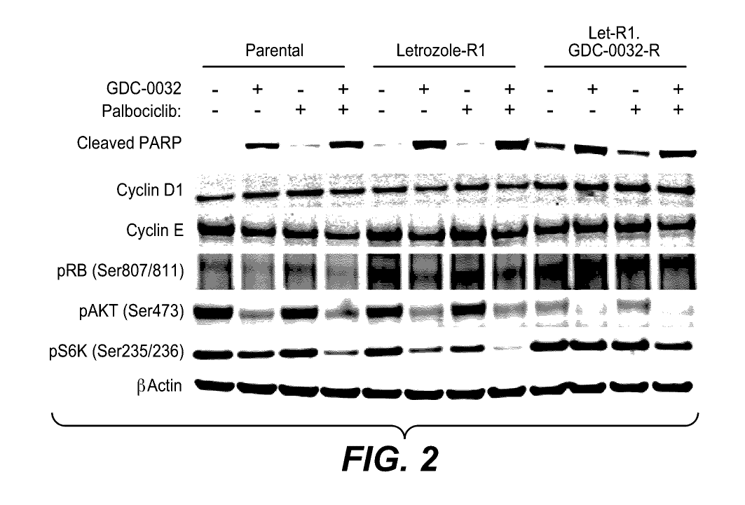

Biomarkers cyclin D1, cyclin E, phosphorylated Rb (Ser807/811) and cleaved PARP were assessed after 24 hours of treatment with taselisib (GDC-0032), palbociclib, and the combination of taselisib+palbociclib (FIG. 2). Cleaved PARP was detected with all taselisib treatments. A decrease in cyclin E was detected with the combination of taselisib and palbociclib. Hyperphosphorylation of Rb at multiple sites, including 807 and 811 is indicative of cells that have entered the cell cycle and are proliferating. Both letrozole resistant (Letrozole-R1) and dual letrozole/taselisib resistant cells (LetR1.GDC-0032-R) had increased phosphorylation of Rb^{Ser807/811} that was decreased with palbociclib and taselisib combination drug treatment. This molecular mechanism is consistent with a recent report using additional PI3K and CDK4/6 inhibitors with MCF7 and T47D parental cells (Vora S R, et al (2014) Cancer cell, 26(1):136-149). As expected, decreases in PI3K pathway signaling were observed with taselisib treatments.

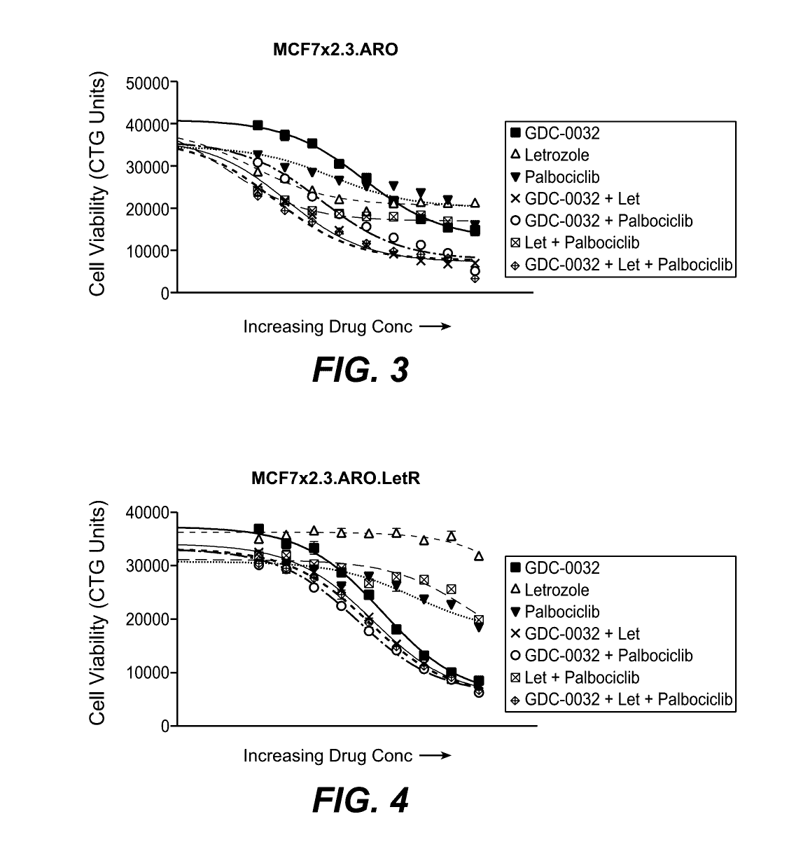

FIG. 3 shows a plot of in vitro cell proliferation data with MCF7×2.3.ARO aromatase expressing breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. The greatest decrease in cell viability appears to come from the GDC-0032+letrozole combination. Similar results are obtained with the triple combination.

FIG. 4 shows a plot of in vitro cell proliferation data with MCF7×2.3.ARO.LetR letrozole-resistant breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. In this letrozole resistant line GDC-0032 potency remains. Any combination that includes GDC-0032 has similar potency to GDC-0032 alone.

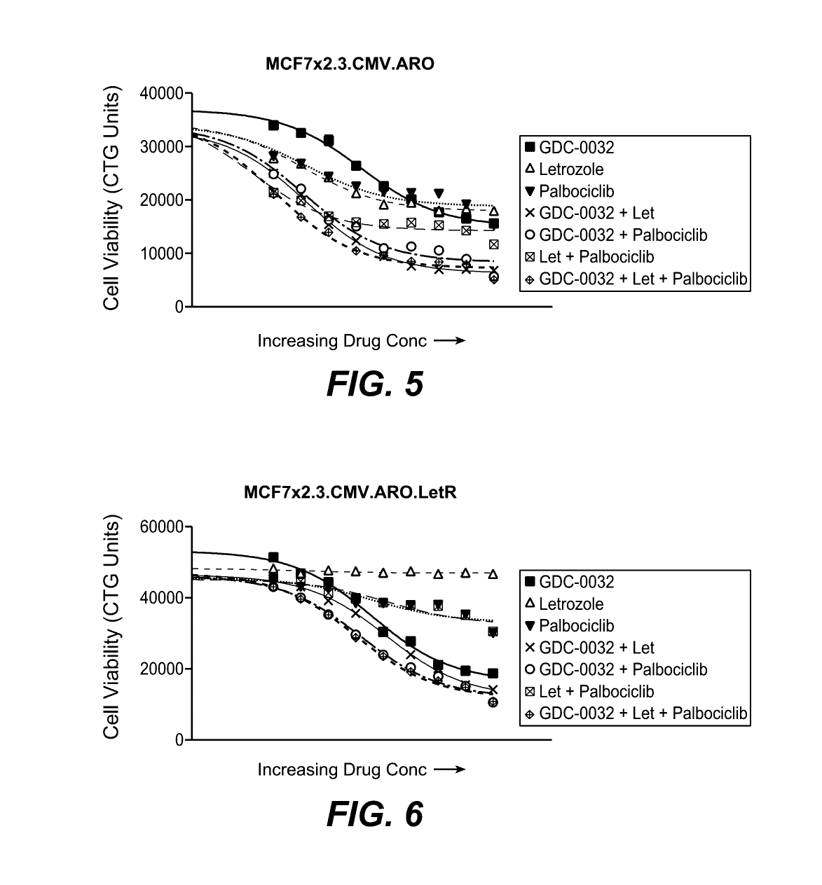

FIG. 5 shows a plot of in vitro cell proliferation data with MCF7×2.3.CMV.ARO breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. The largest contribution to cell viability decrease is observed for the GDC-0032+letrozole combination. Similar results are obtained with the triple combination in this line.

FIG. 6 shows a plot of in vitro cell proliferation data with MCF7×2.3.CMV.ARO.LetR letrozole-resistant breast cancer cells and treatment with dose titrations of: GDC-0032, letrozole, palbociclib, and combinations of GDC-0032+letrozole, GDC-0032+palbociclib, letrozole+palbociclib, and the triple combination of GDC-0032+letrozole+palbociclib. In this letrozole resistant line GDC-0032 potency remains. Any combination that includes GDC-0032 has similar potency to GDC-0032 alone.

These in vitro results suggest that selective PI3K inhibition with GDC-0032, either alone or in combination with palbociclib, is likely to be efficacious in HR+ tumors that are either sensitive or refractory to single agent endocrine therapy such as letrozole treatment.

### Taselisib and Palbociclib Combination In Vivo Tumor Xenograft Activity

GDC-0032 potently inhibits PI3K pathway signaling and combines well with letrozole in an aromatase expressing cell line. In models of letrozole resistance, we found that the PI3K pathway was elevated, but could be diminished by GDC-0032. Moreover, under these conditions of letrozole resistance we found the cells to be equally sensitive to GDC-0032. Letrozole resistant cells were also cultured with a dose escalation of GDC-0032 to derive a model of dual resistance to PI3K/endocrine therapy. Under these conditions, the cells remained equally sensitive to GDC-0032 in combination with a CDK4/6 inhibitor or docetaxel. Taken together, we have developed a model to evaluate the use of PI3K and endocrine therapies in sensitive and refractory ER+ breast cancer cells and demonstrate the activity of a novel inhibitor of class I PI3Ks in this tumor indication.

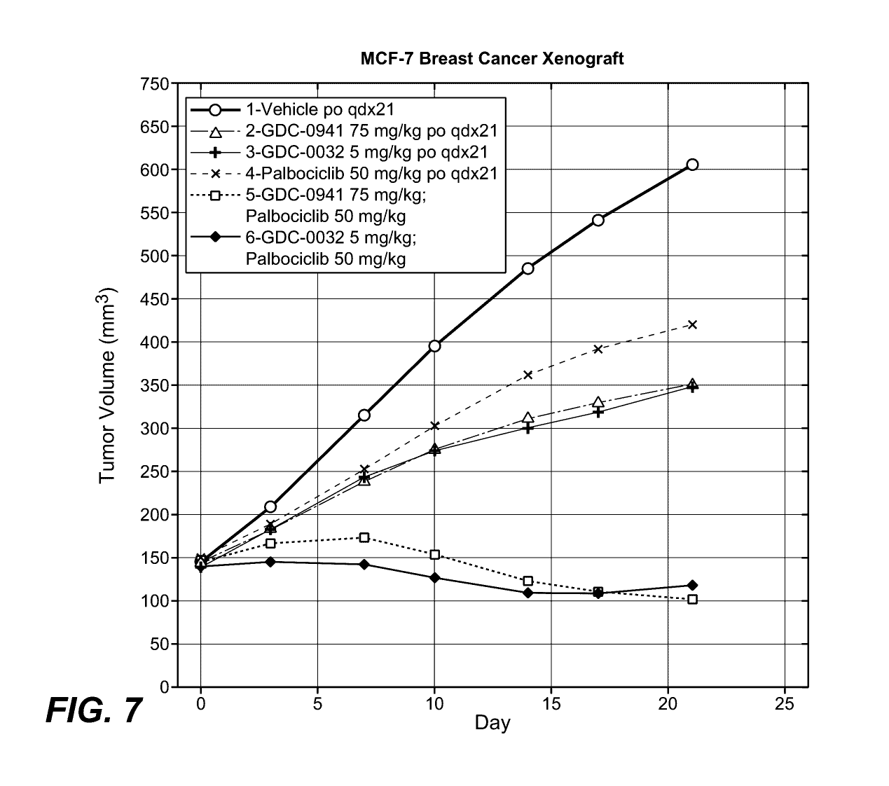

FIG. 7 and Table 1 show the in vivo tumor efficacy study of single agent taselisib, single agent palbociclib, the combination of taselisib and palbociclib, and negative-control vehicle in mice with MCF-7 breast cancer xenografts.

FIG. 7 shows a plot of in vivo tumor volume change over 22 days in cohorts of immunocompromised mice bearing MCF-7 breast cancer xenografts, dosed daily for 21 days by PO (oral) administration with: vehicle, GDC-0941 at 75 mg/kg, taselisib (GDC-0032) at 5 mg/kg, palbociclib at 50 mg/kg, the combination of GDC-0941 (pictilisib) at 75 mg/kg+palbociclib at 50 mg/kg, and the combination of taselisib (GDC-0032) at 5 mg/kg+palbociclib at 50 mg/kg.

TABLE 1

| | Vol | AUC/Day % TGI | TTP | | |
|---|---|---|---|---|---|
| Group | Day 21 | (lower, upper) | 2X | PR | CR |
| 1-vehicle po qdx21 | 605 | 0 (0, 0) | 5.5 | 0 | 0 |
| 3-GDC-0032 5 mg/kg | 349 | 49 (16, 71) | 9.5 | 0 | 0 |
| po qdx21 | | | | | |
| 4-palbociclib 50 mg/kg | 421 | 39 (0, 63) | 8.5 | 0 | 0 |
| po qdx21 | | | | | |
| 6-GDC-0032 5 mg/kg + | 119 | 105 (95, 115) | NA | 2 | 0 |
| palbociclib 50 mg/kg | | | | | |

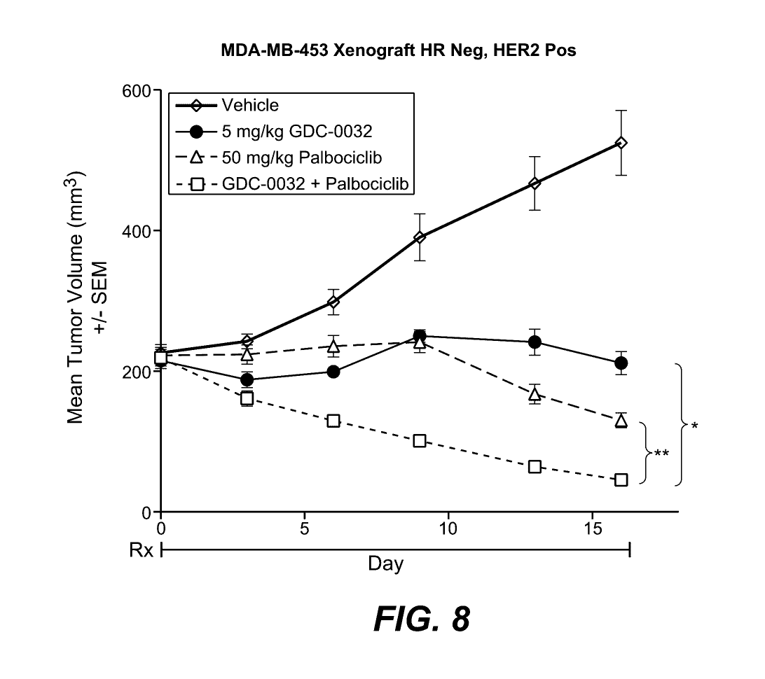

FIG. 8 shows a plot of in vivo tumor volume change over 16 days in cohorts of immunocompromised mice bearing MDA-MB-453 xenografts that is hormone receptor negative (HR neg), HER2 positive (HER2+), and harbors a PIK3CA mutation (H1047R), dosed daily for 21 days by PO (oral) administration with: vehicle, GDC-0032 at 5 mg/kg, palbociclib at 50 mg/kg, and the combination of GDC-0032 at 5 mg/kg+palbociclib at 50 mg/kg.

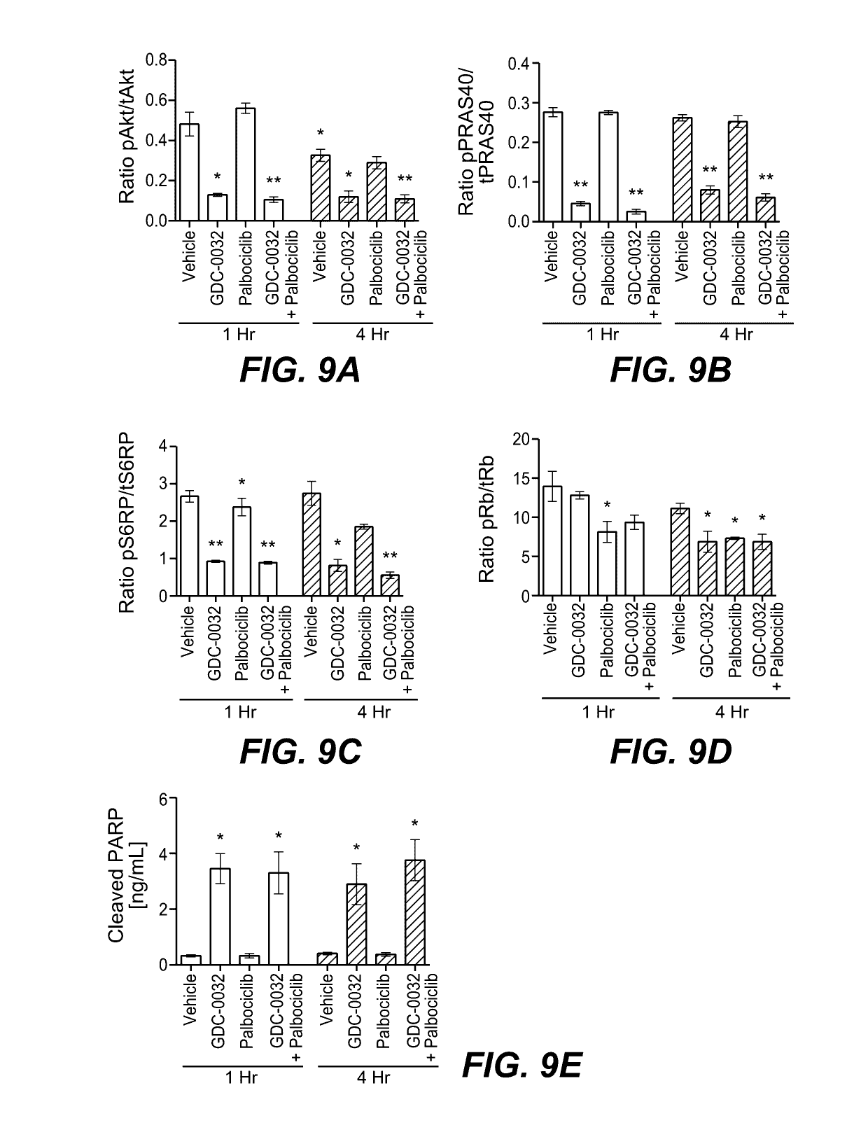

FIGS. 9a-d show ratios of protein levels of mice treated with vehicle, GDC-0032 at 5 mg/kg, palbociclib at 50 mg/kg, and the combination of GDC-0032 at 5 mg/kg+palbociclib at 50 mg/kg, measured at 1 hr and 4 hr. FIG. 9a shows the ratio of phosphoAkt (pAkt) to total Akt (tAkt). FIG. 9b shows the ratio of phospho PRAS40 (pPRAS40) to total PRAS40 (tPRAS40). FIG. 9c shows the ratio of phospho S6RP (pS6RP) to total S6RP (tS6RP). FIG. 9d shows the ratio of phosphor Rb (pRb) to total Rb (tRb).

FIG. 9e shows the concentration of cleaved PARP [ng/mL] of mice treated with vehicle, GDC-0032 at 5 mg/kg, palbociclib at 50 mg/kg, and the combination of GDC-0032 at 5 mg/kg+palbociclib at 50 mg/kg, measured at 1 hr and 4 hr.

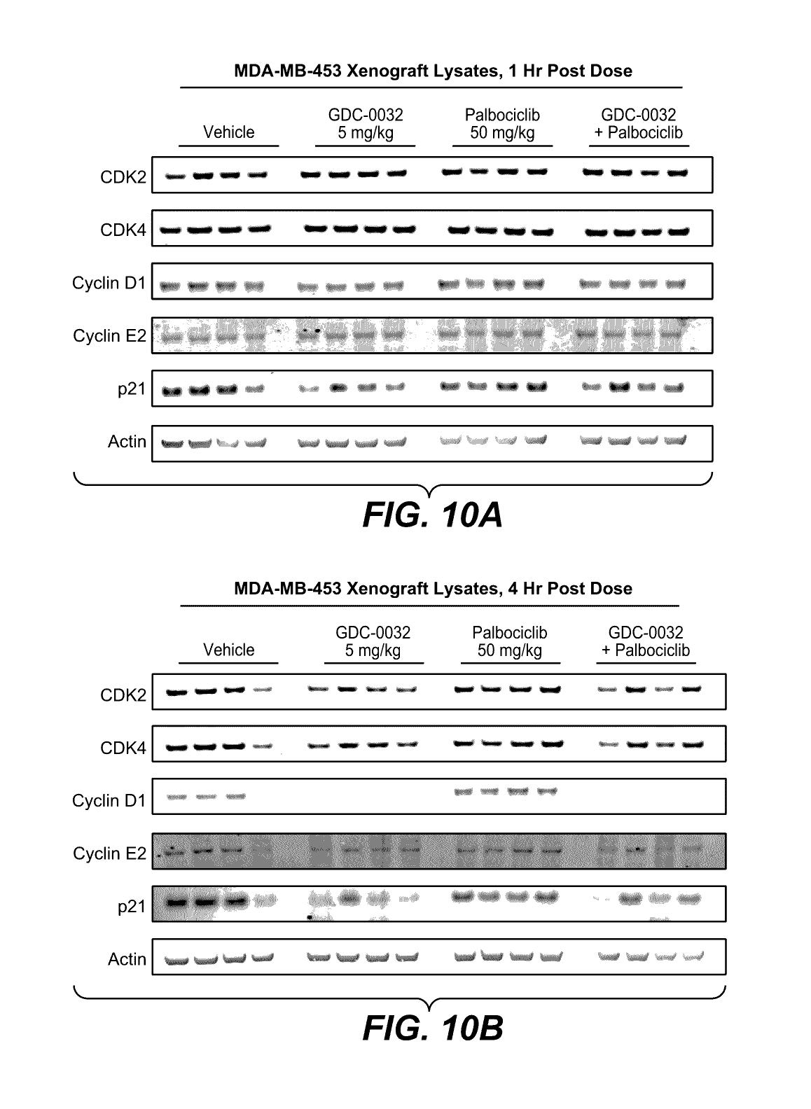

FIGS. 10a and 10b shows pathway signaling effects by western blot autoradiograms of gel electrophoresis of cell lysates collected after 1 hour (FIG. 10a) and 4 hours (FIG. 10b) of exposure to no drug (Vehicle), GDC-0032 at 5 mg/kg, palbociclib at 50 mg/kg, and the combination of GDC-0032+palbociclib in the MDA-MB-453 xenograft. Levels of CDK2, CDK4, cyclin D1, cyclin E2, p21, and Actin were visualized.

The combination of GDC-0032 (taselisib) with palbociclib results in increased tumor growth inhibition and tumor regressions in the MDA-MB-453 HR-/HER2+ xenograft model when compared to each drug alone. Notably, the enhanced efficacy of palbociclib when combined with GDC-0032 is in the MDA-MB-453 tumor model, shown in FIG. 8, which harbors the H1047R hotspot PI3K mutation in PIK3CA (p110a). GDC-0032 effectively decreased levels of PI3K pathway markers such as pAkt (FIG. 9a), pPRAS40 (FIG. 9b) and pS6RP (FIG. 9c) in MDA-MB-453 tumors that were elevated due to increased pathway activation as a result of the PIK3CA mutation and HER2 over-expression. The latter pharmacodynamic effects corroborates that GDC-0032 was tested at pharmacologically active doses. Both GDC-0032 and palbociclib decreased levels of pRB (FIG. 9d) in MDA-MB-453 tumors demonstrating that both drugs blocked cells in G1 of the cell-cycle as predicted based on their mechanism of action and confirmed that palbociclib was also tested at pharmacologically active doses. Lastly, based on a unique PIK3CA mutant-selective mechanism of action, GDC-0032 induced cell death (based on increased cleaved PARP) in MDA-MB-453 tumors (FIG. 9e) confirming that this model is dependent on the PIK3CA mutation for growth and is sensitive to PI3K inhibition.

### Pharmaceutical Compositions and Formulations

Pharmaceutical compositions or formulations of the present invention include the therapeutic combination of taselisib and palbociclib, and one or more pharmaceutically acceptable carrier, glidant, diluent, or excipient.

Taselisib and palbociclib may exist in unsolvated as well as solvated forms with pharmaceutically acceptable solvents such as water, ethanol, and the like, and it is intended that the invention embrace both solvated and unsolvated forms.

The compounds of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Pharmaceutical compositions encompass both the bulk composition and individual dosage units comprised of more than one (e.g., two) pharmaceutically active agents including the therapeutic combinations of taselisib and palbociclib described herein, along with any pharmaceutically inactive excipients, diluents, carriers, or glidants. The bulk composition and each individual dosage unit can contain fixed amounts of the aforesaid pharmaceutically active agents. The bulk composition is material that has not yet been formed into individual dosage units. An illustrative dosage unit is an oral dosage unit such as tablets, pills, capsules, and the like. Similarly, the methods of treating a patient by administering a pharmaceutical composition is also intended to encompass the administration of the bulk composition and individual dosage units.

Pharmaceutical compositions also embrace isotopically-labeled forms of taselisib and palbociclib which are identical to those recited herein, but for the fact that one or more atoms are replaced by an atom having an atomic mass or mass number different from the atomic mass or mass number usually found in nature. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Examples herein below, by substituting an isotopically labeled reagent for a non-isotopically labeled reagent.

Taselisib and palbociclib are formulated in accordance with standard pharmaceutical practice for use in a therapeutic combination for therapeutic treatment (including prophylactic treatment) of hyperproliferative disorders in mammals including humans. The invention provides a pharmaceutical composition comprising taselisib and palbociclib in association with one or more pharmaceutically acceptable carrier, glidant, diluent, additive, or excipient.

Suitable carriers, diluents, additives, and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), dimethylsulfoxide (DMSO), cremophor (e.g. CREMOPHOR EL®, BASF), and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, taselisib and palbociclib having the desired degree of purity may optionally be mixed with pharmaceutically acceptable diluents, carriers, excipients or stabilizers (Remington's Pharmaceutical Sciences (1995) 18th edition, Mack Publ. Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations of the invention will be dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

The initial pharmaceutically effective amounts of taselisib and palbociclib administered orally or parenterally per dose will be in the range of about 0.01-1000 mg/kg, namely about 0.1 to 20 mg/kg of patient body weight per day, with the typical initial range of compound used being 0.3 to 15 mg/kg/day. The doses of taselisib and palbociclib to be administered may range for each from about 1 mg to about 1000 mg per unit dosage form, or from about 10 mg to about 100 mg per unit dosage form. The doses of taselisib and palbociclib may be administered in a ratio of about 1:50 to about 50:1 by weight, or in a ratio of about 1:10 to about 10:1 by weight.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, CREMOPHOR EL®, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, Pa.

Sustained-release preparations of taselisib and palbociclib may be prepared. Suitable examples of sustained-release preparations include semipermeable matrices of solid hydrophobic polymers, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D (−) 3-hydroxybutyric acid.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, Pa. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of taselisib and palbociclib suitable for oral administration may be prepared as discrete units such as pills, hard or soft e.g., gelatin capsules, cachets, troches, lozenges, aqueous or oil suspensions, dispersible powders or granules, emulsions, syrups or elixirs each containing a predetermined amount of GDC-0032 and/or a chemotherapeutic agent. The amount of GDC-0032 and the amount of chemotherapeutic agent may be formulated in a pill, capsule, solution or suspension as a combined formulation. Alternatively, GDC-0032 and the chemotherapeutic agent may be formulated separately in a pill, capsule, solution or suspension for administration by alternation.

Formulations may be prepared according to any method known to the art for the manufacture of pharmaceutical compositions and such compositions may contain one or more agents including sweetening agents, flavoring agents, coloring agents and preserving agents, in order to provide a palatable preparation. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablet excipients of a pharmaceutical formulation of the invention may include: Filler (or diluent) to increase the bulk volume of the powdered drug making up the tablet; Disintegrants to encourage the tablet to break down into small fragments, ideally individual drug particles, when it is ingested and promote the rapid dissolution and absorption of drug; Binder to ensure that granules and tablets can be formed with the required mechanical strength and hold a tablet together after it has been compressed, preventing it from breaking down into its component powders during packaging, shipping and routine handling; Glidant to improve the flowability of the powder making up the tablet during production; Lubricant to ensure that the tabletting powder does not adhere to the equipment used to press the tablet during manufacture. They improve the flow of the powder mixes through the presses and minimize friction and breakage as the finished tablets are ejected from the equipment; Antiadherent with function similar to that of the glidant, reducing adhesion between the powder making up the tablet and the machine that is used to punch out the shape of the tablet during manufacture; Flavor incorporated into tablets to give them a more pleasant taste or to mask an unpleasant one, and Colorant to aid identification and patient compliance.

Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

The aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner, including a mixture of at least one emulsifier with a fat or an oil, or with both a fat and an oil. Preferably, a hydrophilic emulsifier is included together with a lipophilic emulsifier which acts as a stabilizer. Together, the emulsifier(s) with or without stabilizer(s) make up an emulsifying wax, and the wax together with the oil and fat comprise an emulsifying ointment base which forms the oily dispersed phase of cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of the pharmaceutical formulations of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

Therapeutic combinations of taselisib and palbociclib may be employed in combination with certain chemotherapeutic agents for the treatment of a hyperproliferative disorder, including solid tumor cancer types or hematopoietic malignancy, along with pre-malignant and non-neoplastic or non-malignant hyperproliferative disorders. Therapeutic combinations of taselisib and palbociclib may be further employed in combination with certain chemotherapeutic agents in a “cocktail” or other dosing regimen to treat cancer. In certain embodiments, taselisib and palbociclib are combined in a single formulation (co-formulated) as a single tablet, pill, capsule, or solution for simultaneous administration of the combination. In other embodiments, taselisib and palbociclib are administered according to a dosage regimen or course of therapy in separate formulations as separate tablets, pills, capsules, or solutions for sequential or coincidental administration of taselisib and palbociclib. The combination of taselisib and palbociclib may have synergistic properties. The therapeutic combination taselisib and palbociclib may be administered in amounts that are effective for the purpose intended. In one embodiment, a pharmaceutical formulation of this invention comprises taselisib and palbociclib. In another embodiment, the therapeutic combination is administered by a dosing regimen wherein the therapeutically effective amount of taselisib is administered in a range from twice daily to once every three weeks (q3wk), and the therapeutically effective amount of palbociclib is administered separately, in alternation, in a range from twice daily to once every three weeks.

Therapeutic combinations of the invention include taselisib and palbociclib for separate, simultaneous or sequential use in the treatment of a hyperproliferative disorder such as cancer.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments, such as to increase the therapeutic index or mitigate toxicity or other side-effects or consequences.

In a particular embodiment of anti-cancer therapy, the therapeutic combination may be combined with surgical therapy and radiotherapy, as adjuvant therapy. Combination therapies according to the present invention include the administration of a combination of taselisib and palbociclib, and one or more other cancer treatment methods or modalities. The amounts of taselisib and palbociclib and the relative timings of administration will be selected in order to achieve the desired combined therapeutic effect.

### Administration of Pharmaceutical Compositions

Therapeutic combinations of taselisib and palbociclib may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. Formulation of drugs is discussed in Remington's Pharmaceutical Sciences, 18^{th} Ed., (1995) Mack Publishing Co., Easton, Pa. Other examples of drug formulations can be found in Liberman, H. A. and Lachman, L., Eds., Pharmaceutical Dosage Forms, Marcel Decker, Vol 3, 2^{nd} Ed., New York, N.Y. For local immunosuppressive treatment, the compounds may be administered by intralesional administration, including perfusing or otherwise contacting the graft with the inhibitor before transplantation. It will be appreciated that the preferred route may vary with for example the condition of the recipient. Where a compound of the therapeutic combination is administered orally, it may be formulated as a pill, capsule, tablet, etc. with a pharmaceutically acceptable carrier, glidant, or excipient. Where the compound of the therapeutic combination is administered parenterally, it may be formulated with a pharmaceutically acceptable parenteral vehicle or diluent, and in a unit dosage injectable form, as detailed below.

A dose to treat human patients may range from about 1 mg to about 1000 mg of each of taselisib and palbociclib, such as about 3 mg to about 200 mg of the compound. A dose may be administered once a day (QD), twice per day (BID), or more frequently, depending on the pharmacokinetic (PK) and pharmacodynamic (PD) properties, including absorption, distribution, metabolism, and excretion of the particular compound. In addition, toxicity factors may influence the dosage and administration dosing regimen. When administered orally, the pill, capsule, or tablet may be ingested twice daily, daily or less frequently such as weekly or once every two or three weeks for a specified period of time. The regimen may be repeated for a number of cycles of therapy.

### Methods of Treatment

The methods of the invention include:
methods of diagnosis based on the identification of a biomarker;
methods of determining whether a patient will respond to a therapeutic combination of taselisib and palbociclib;
methods of optimizing therapeutic efficacy by monitoring clearance of taselisib, palbociclib, or a combination of taselisib and palbociclib;
methods of optimizing a therapeutic regimen of a therapeutic combination of taselisib and palbociclib, by monitoring the development of therapeutic resistance mutations; and
methods for identifying which patients will most benefit from treatment with a therapeutic combination of taselisib and palbociclib, and monitoring patients for their sensitivity and responsiveness to treatment with the therapeutic combination of taselisib and palbociclib.

The methods of the invention are useful for inhibiting abnormal cell growth or treating a hyperproliferative disorder such as cancer in a mammal (e.g., a human patient with a hyperproliferative disorder such as cancer). For example, the methods are useful for diagnosing, monitoring, and treating multiple myeloma, lymphoma, leukemias, prostate cancer, breast cancer, hepatocellular carcinoma, pancreatic cancer, and/or colorectal cancer in a mammal (e.g., human).

Therapeutic combinations of taselisib and palbociclib are useful for treating diseases, conditions and/or disorders including, but not limited to, those characterized by activation of the PI3 kinase pathway. Accordingly, another aspect of this invention includes methods of treating diseases or conditions that can be treated by inhibiting lipid kinases, including PI3. In one embodiment, a method for the treatment of a solid tumor or hematopoietic malignancy comprises administering a therapeutic combination as a combined formulation or by alternation to a mammal, wherein the therapeutic combination comprises a therapeutically effective amount of taselisib, and a therapeutically effective amount of palbociclib. Therapeutic combinations of taselisib and palbociclib may be employed for the treatment of a hyperproliferative disease or disorder, including hematopoietic malignancy, tumors, cancers, and neoplastic tissue, along with pre-malignant and non-neoplastic or non-malignant hyperproliferative disorders. In one embodiment, a human patient is treated with a therapeutic combination and a pharmaceutically acceptable carrier, adjuvant, or vehicle, wherein taselisib, or metabolite thereof, of said therapeutic combination is present in an amount to detectably inhibit PI3 kinase activity.

Hematopoietic malignancies include non-Hodgkin's lymphoma, diffuse large hematopoietic lymphoma, follicular lymphoma, mantle cell lymphoma, chronic lymphocytic leukemia, multiple myeloma, AML, and MCL.

Another aspect of this invention provides a pharmaceutical composition or therapeutic combination for use in the treatment of the diseases or conditions described herein in a mammal, for example, a human patient, suffering from such disease or condition. Also provided is the use of a pharmaceutical composition in the preparation of a medicament for the treatment of the diseases and conditions described herein in a warm-blooded animal, such as a mammal, for example a human patient, suffering from such disorder.

### Articles of Manufacture

In another embodiment of the invention, an article of manufacture, or “kit”, containing taselisib and palbociclib useful for the treatment of the diseases and disorders described above is provided. In one embodiment, the kit comprises a container comprising taselisib and palbociclib. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold taselisib and palbociclib, or a co-formulation thereof, which is effective for treating the condition and may have a sterile access port (for example, the container may be an intravenous solution bag or a vial having a stopper pierceable by a hypodermic injection needle). The label or package insert indicates that the contents are used for treating the condition of choice, such as cancer. In one embodiment, the label or package inserts indicates that the therapeutic combination of taselisib and palbociclib can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of taselisib and palbociclib. For example, if the kit comprises a first composition comprising taselisib and a second composition comprising palbociclib, the kit may further comprise directions for the simultaneous, sequential or separate administration of the first and second pharmaceutical compositions to a patient in need thereof.

In another embodiment, the kits are suitable for the delivery of solid oral forms of taselisib and palbociclib, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with taselisib contained therein; and (b) a second container with palbociclib contained therein. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises taselisib and palbociclib, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

### Example 1

### p110a (Alpha) PI3K Binding Assay

Binding Assays: Initial polarization experiments were performed on an Analyst HT 96-384 (Molecular Devices Corp, Sunnyvale, Calif.). Samples for fluorescence polarization affinity measurements were prepared by addition of 1:3 serial dilutions of p110alpha PI3K (Upstate Cell Signaling Solutions, Charlottesville, Va.) starting at a final concentration of 20 ug/mL in polarization buffer (10 mM Tris pH 7.5, 50 mM NaCl, 4 mM MgCl₂, 0.05% Chaps, and 1 mM DTT) to 10 mM PIP₂ (Echelon-Inc., Salt Lake City, Utah) final concentration. After an incubation time of 30 minutes at room temperature, the reactions were stopped by the addition of GRP-1 and PIP3-TAMRA probe (Echelon-Inc., Salt Lake City, Utah) 100 nM and 5 nM final concentrations respectively. Read with standard cut-off filters for the rhodamine fluorophore (λex=530 nm; λem=590 nm) in 384-well black low volume Proxiplates® (PerkinElmer, Wellesley, Mass.) Fluorescence polarization values were plotted as a function of the protein concentration. EC₅₀ values were obtained by fitting the data to a four-parameter equation using KaleidaGraph® software (Synergy software, Reading, Pa.). This experiment also establishes the appropriate protein concentration to use in subsequent competition experiments with inhibitors.

Inhibitor IC₅₀ values were determined by addition of the 0.04 mg/mL p110alpha PI3K (final concentration) combined with PIP₂ (10 mM final concentration) to wells containing 1:3 serial dilutions of the antagonists in a final concentration of 25 mM ATP (Cell Signaling Technology, Inc., Danvers, Mass.) in the polarization buffer. After an incubation time of 30 minutes at room temperature, the reactions were stopped by the addition of GRP-1 and PIP3-TAMRA probe (Echelon-Inc., Salt Lake City, Utah) 100 nM and 5 nM final concentrations respectively. Read with standard cut-off filters for the rhodamine fluorophore (λex=530 nm; λem=590 nm) in 384-well black low volume Proxiplates® (PerkinElmer, Wellesley, Mass.) Fluorescence polarization values were plotted as a function of the antagonist concentration, and the IC₅₀ values were obtained by fitting the data to a 4-parameter equation in Assay Explorer software (MDL, San Ramon, Calif.).

Alternatively, inhibition of PI3K was determined in a radiometric assay using purified, recombinant enzyme and ATP at a concentration of 1 μM (micromolar). The compound was serially diluted in 100% DMSO. The kinase reaction was incubated for 1 h at room temperature, and the reaction was terminated by the addition of PBS. IC₅₀ values were subsequently determined using sigmoidal dose-response curve fit (variable slope).

### Example 2

### In Vitro Cell Proliferation Assays

Cell culture. MCF7 cell line was obtained from the American Type Culture Collection (ATCC, VA). The cells were tested and authenticated using gene expression and single nucleotide polymorphism genotyping arrays (Hoeflich K P, et al (2009) Clin Cancer Res, 15(14):4649-4664; Hu X, et al (2009) Mol Cancer Res, 7(4):511-522) and cultured in RPMI supplemented with 10% fetal bovine serum, 100 units/ml penicillin, 100 □g/ml streptomycin, 2 mM L-glutamine and NEAA at 37° C. under 5% CO₂. Stable aromatase-expressing MCF7 cells (MCF7-ARO) were generated by transfection of a plasmid vector containing the full aromatase gene and a neomycin selection gene. The cells were maintained in androstenedione and all experiments were performed in the presence of androstenedione except where indicated

Efficacy of GDC-0032 and chemotherapeutic compounds were measured by a cell proliferation assay employing the following protocol (Mendoza et al (2002) Cancer Res. 62:5485-5488).

The CellTiter-Glo® Luminescent Cell Viability Assay is a homogeneous method to determine the number of viable cells in culture based on quantitation of the ATP present, which signals the presence of metabolically active cells. The CellTiter-Glo® Assay is designed for use with multiwell plate formats, making it ideal for automated high-throughput screening (HTS), cell proliferation and cytotoxicity assays. The homogeneous assay procedure involves adding a single reagent (CellTiter-Glo® Reagent) directly to cells cultured in serum-supplemented medium. Cell washing, removal of medium or multiple pipetting steps are not required. The Cell Titer-Glo® Luminescent Cell Viability Assay, including reagents and protocol are commercially available (Promega Corp., Madison, Wis., Technical Bulletin TB288).

The assay assesses the ability of compounds to enter cells and inhibit cell proliferation. The assay principle is based on the determination of the number of viable cells present by quantitating the ATP present in a homogenous assay where addition of the Cell Titer-Glo® reagent results in cell lysis and generation of a luminescent signal through the luciferase reaction. The luminescent signal is proportional to the amount of ATP present.

Procedure: Day 1—Seed Cell Plates (384-well black, clear bottom, microclear, TC plates with lid from Falcon #353962), Harvest cells, Seed cells at 1000 cells per 54 μl per well into 384 well Cell Plates for 3 days assay. Cell Culture Medium: RPMI or DMEM high glucose, 10% Fetal Bovine Serum, 2 mM L-Glutamine, P/S. Incubate 0/N (overnight) at 37° C., 5% CO₂.

Cell viability assays. 384-well plates were seeded with 2000 cells/well in a volume of 54 μl per well followed by incubation at 37° C. under 5% CO₂ overnight (˜16 hours). Compounds were diluted in DMSO to generate the desired stock concentrations then added in a volume of 6 μL per well. All treatments were tested in quadruplicate. After 4 days incubation, relative numbers of viable cells were estimated using CellTiter-Glo (Promega, Madison, Wis.) and total luminescence was measured on an Envision plate Reader (PerkinElmer, Foster City, Calif.). The concentration of drug resulting in 50% inhibition of cell viability (IC₅O) or 50% maximal effective concentration (EC₅₀) was determined using Prism software (GraphPad, La Jolla, Calif.)

Day 2—Add Drug to Cells, Compound Dilution, DMSO Plates (serial 1:2 for 9 points). Add 20 μl of compound at 10 mM in the 2nd column of 96 well plate. Perform serial 1:2 across the plate (10 μl+20 μl 100% DMSO) for a total of 9 points using Precision Media Plates 96-well conical bottom polypropylene plates from Nunc (cat.#249946) (1:50 dilution). Add 147 μl of Media into all wells. Transfer 3 μl of DMSO+compound from each well in the DMSO Plate to each corresponding well on Media Plate using Rapidplate® (Caliper, a Perkin-Elmer Co.). For 2 drug combination studies, transfer one drug 1.5 μl of DMSO+compound from each well in the DMSO Plate to each corresponding well on Media Plate using Rapidplate. Then, transfer another drug 1.5 μl to the medium plate.

Drug Addition to Cells, Cell Plate (1:10 dilution): Add 6 μl of media+compound directly to cells (54 μl of media on the cells already). Incubate 3 days at 37° C., 5% CO₂ in an incubator that will not be opened often.

Day 5—Develop Plates, Thaw Cell Titer Glo Buffer at room temperature: Remove Cell Plates from 37° C. and equilibrate to room temperature for about 30 minutes. Add Cell Titer-Glo® Buffer to Cell Titer-Glo® Substrate (bottle to bottle). Add 30 μl Cell Titer-Glo® Reagent (Promega cat.# G7572) to each well of cells. Place on plate shaker for about 30 minutes. Read luminescence on Analyst HT Plate Reader (half second per well).

Cell viability assays and combination assays: Cells were seeded at 1000-2000 cells/well in 384-well plates for 16 h. On day two, nine serial 1:2 compound dilutions were made in DMSO in a 96 well plate. The compounds were further diluted into growth media using a Rapidplate® robot (Zymark Corp., Hopkinton, Mass.). The diluted compounds were then added to quadruplicate wells in 384-well cell plates and incubated at 37° C. and 5% CO₂. After 4 days, relative numbers of viable cells were measured by luminescence using Cell Titer-Glo® (Promega) according to the manufacturer's instructions and read on a Wallac Multilabel Reader® (PerkinElmer, Foster City). EC50 values were calculated using Prism® 4.0 software (GraphPad, San Diego). Drugs in combination assays were dosed starting at 4×EC₅₀ concentrations. If cases where the EC50 of the drug was >2.5 the highest concentration used was 10 μM. GDC-0032 and chemotherapeutic agents were added simultaneously or separated by 4 hours (one before the other) in all assays.

Letrozole resistant cell line selection. MCF7-ARO cells were grown in increasing concentrations of letrozole in the presence of androstenedione in phenol red free RPMI medium, supplement with 10% Charcoal dextran stripped FBS, until they grew normally in a letrozole concentration of 6.5 μmol/L. For cells resistant to both letrozole and GDC-0032, letrozole resistant cells were grown in increasing concentrations of the GDC-0032, until they grew normally in a concentration of 2.5 μmol/L. Maintenance of aromatase expression in all letrozole sensitive and resistant clones was verified using TaqMan.

An additional exemplary in vitro cell proliferation assay includes the following steps:

1. An aliquot of 100 μl of cell culture containing about 10⁴ cells (see Table 3 for cell lines and tumor type) in medium was deposited in each well of a 384-well, opaque-walled plate.

2. Control wells were prepared containing medium and without cells.

3. The compound was added to the experimental wells and incubated for 3-5 days.

4. The plates were equilibrated to room temperature for approximately 30 minutes.

5. A volume of CellTiter-Glo® Reagent equal to the volume of cell culture medium present in each well was added.

6. The contents were mixed for 2 minutes on an orbital shaker to induce cell lysis.

7. The plate was incubated at room temperature for 10 minutes to stabilize the luminescence signal.

8. Luminescence was recorded and reported in graphs as RLU=relative luminescence units.

9. Analyze using the Chou and Talalay combination method and Dose-Effect Analysis with CalcuSyn® software (Biosoft, Cambridge, UK) in order to obtain a Combination Index.

Alternatively, cells were seeded at optimal density in a 96 well plate and incubated for 4 days in the presence of test compound. Alamar Blue™ was subsequently added to the assay medium, and cells were incubated for 6 h before reading at 544 nm excitation, 590 nm emission. EC₅₀ values were calculated using a sigmoidal dose response curve fit.

Alternatively, Proliferation/Viability was analyzed after 48 hr of drug treatment using Cell Titer-Glo® reagent (Promega Inc., Madison, Wis.). DMSO treatment was used as control in all viability assays. IC₅₀ values were calculated using XL fit software (IDBS, Alameda, Calif.)

The cell lines were obtained from either ATCC (American Type Culture Collection, Manassas, Va.) or DSMZ (Deutsche Sammlung von Mikroorganismen and Zellkulturen GmbH, Braunschweig, DE). Cells were cultured in RPMI 1640 medium supplemented with 10% fetal bovine serum, 100 units/ml penicillin, 2 mM L-glutamine, and 100 mg/ml streptomycin (Life Technology, Grand Island, N.Y.) at 37° C. under 5% CO₂.

Letrozole (FEMARA®, Novartis Pharm.) is an oral non-steroidal aromatase inhibitor for the treatment of hormonally-responsive breast cancer after surgery (Bhatnagar et al (1990) J. Steroid Biochem. and Mol. Biol. 37:1021; Lipton et al (1995) Cancer 75:2132; Goss, P. E. and Smith, R. E. (2002) Expert Rev. Anticancer Ther. 2:249-260; Lang et al (1993) The Journal of Steroid Biochem. and Mol. Biol. 44 (4-6):421-8; EP 236940; U.S. Pat. No. 4,978,672). FEMARA® is approved by the FDA for the treatment of local or metastatic breast cancer that is hormone receptor positive (HR+) or has an unknown receptor status in postmenopausal women. Letrozole is named as 4,4′-((1H-1,2,4-triazol-1-yl)methylene)dibenzonitrile (CAS Reg. No. 112809-51-5), and has the structure:

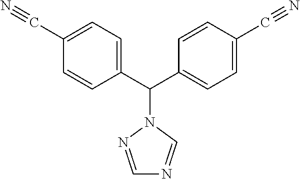 [SMILES: N#Cc1ccc(C(c2ccc(C#N)cc2)n2cncn2)cc1]

### Example 3

### In Vivo Mouse Tumor Xenograft Efficacy

Mice: Female severe combined immunodeficiency mice (Fox Chase SCID®, C.B-17/IcrHsd, Harlan) or nude mice (Taconic Farms, Harlan) were 8 to 9 weeks old and had a BW range of 15.1 to 21.4 grams on Day 0 of the study. The animals were fed ad libitum water (reverse osmosis, 1 ppm Cl) and NIH 31 Modified and Irradiated Lab Diet® consisting of 18.0% crude protein, 5.0% crude fat, and 5.0% crude fiber. The mice were housed on irradiated ALPHA-Dri® Bed-o'Cobs® Laboratory Animal Bedding in static microisolators on a 12-hour light cycle at 21-22° C. (70-72° F.) and 40-60% humidity. PRC specifically complies with the recommendations of the Guide for Care and Use of Laboratory Animals with respect to restraint, husbandry, surgical procedures, feed and fluid regulation, and veterinary care. The animal care and use program at PRC is accredited by the Association for Assessment and Accreditation of Laboratory Animal Care International (AAALAC), which assures compliance with accepted standards for the care and use of laboratory animals.

Tumor Implantation:

Xenografts were initiated with cancer cells, including breast cancer cell lines MCF-7 (Soule H. D. et al (1973) Jour. Nat. Cancer Inst. 51 (5): 1409-1416; Levenson A. S. et al (1997) Cancer Res. 57 (15): 3071-3078; LaCroix M. et al (2004) Breast Res. and Treatment 83 (3): 249-289) and MDA-MB-453 (Vranic S. et al (2011) Onc. Letters 2: 1131-1137; Hall R. E. et al (1994) Euro. Jour. Cancer 30(4):484-490). Cells were cultured in RPMI 1640 medium supplemented with 10% fetal bovine serum, 2 mM glutamine, 100 units/mL penicillin, 100 μl/mL streptomycin sulfate and 25 μg/mL gentamicin. The cells were harvested during exponential growth and resuspended in phosphate buffered saline (PBS) at a concentration of 5×10⁶ or 10×10⁶ cells/mL depending on the doubling time of the cell line. Tumor cells were implanted subcutaneously in the right flank, and tumor growth was monitored as the average size approached the target range of 100 to 150 mm3. Twenty-one days after tumor implantation, designated as Day 0 of the study, the mice were placed into four groups each consisting of ten mice with individual tumor volumes ranging from 75-172 mm3 and group mean tumor volumes from 120-121 mm3 (see Appendix A). Volume was calculated using the formula:

Tumor Volume (mm³)=(w²×l)/2,

where w=width and l=length in mm of a tumor. Tumor weight may be estimated with the assumption that 1 mg is equivalent to 1 mm3 of tumor volume.

Therapeutic Agents:

GDC-0032 was supplied as a dry powder in salt form, which contained 73% active agent, and was stored at room temperature protected from light. Drug doses were prepared weekly in 0.5% methylcellulose: 0.2% Tween 80 in deionized water (“Vehicle”) and stored at 4° C. The salt form containing 73% active agent was accounted for in the formulation of GDC-0032 doses. Doses of GDC-0032 were prepared on each day of dosing by diluting an aliquot of the stock with sterile saline (0.9% NaCl). All doses were formulated to deliver the stated mg/kg dosage in a volume of 0.2 mL per 20 grams of body weight (10 mL/kg).

Treatment:

All doses were scaled to the body weights of the individual animals and were provided by the route indicated in each of the figures.

Endpoint:

Tumor volume was measured in 2 dimensions (length and width), using Ultra Cal IV calipers (Model 54 10 111; Fred V. Fowler Company), as follows: tumor volume (mm³)=(length×width²)×0.5 and analyzed using Excel version 11.2 (Microsoft Corporation). A linear mixed effect (LME) modeling approach was used to analyze the repeated measurement of tumor volumes from the same animals over time (Pinheiro, J. et al (2009); Tan, N. et al (2011) Clin. Cancer Res. 17(6):1394-1404). This approach addresses both repeated measurements and modest dropouts due to any non-treatment-related death of animals before study end. Cubic regression splines were used to fit a nonlinear profile to the time courses of log 2 tumor volume at each dose level. These nonlinear profiles were then related to dose within the mixed model. Tumor growth inhibition as a percentage of vehicle control (% TGI) was calculated as the percentage of the area under the fitted curve (AUC) for the respective dose group per day in relation to the vehicle, using the following formula: % TGI=100×(1−AUC_{dose}/AUCᵥₑₕ). Using this formula, a TGI value of 100% indicates tumor stasis, a TGI value of >1% but <100% indicates tumor growth delay, and a TGI value of >100% indicates tumor regression. Partial response (PR) for an animal was defined as a tumor regression of >50% but <100% of the starting tumor volume. Complete response (CR) was defined as 100% tumor regression (i.e., no measurable tumor) on any day during the study.

Toxicity:

Animals were weighed daily for the first five days of the study and twice weekly thereafter. Animal body weights were measured using an Adventurer Pro® AV812 scale (Ohaus Corporation). Percent weight change was calculated as follows: body weight change (%)=[(weight_{day new}−weight_{day 0})/weight_{day 0}]×100. The mice were observed frequently for overt signs of any adverse, treatment-related side effects, and clinical signs of toxicity were recorded when observed. Acceptable toxicity is defined as a group mean body weight (BW) loss of less than 20% during the study and not more than one treatment-related (TR) death among ten treated animals. Any dosing regimen that results in greater toxicity is considered above the maximum tolerated dose (MTD). A death is classified as TR if attributable to treatment side effects as evidenced by clinical signs and/or necropsy, or may also be classified as TR if due to unknown causes during the dosing period or within 10 days of the last dose. A death is classified as NTR if there is no evidence that death was related to treatment side effects.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

We claim:

1. A method for the treatment of cancer comprising administering a therapeutic combination as a combined formulation or by alternation to a patient, wherein the therapeutic combination comprises a therapeutically effective amount of taselisib, and a therapeutically effective amount of palbociclib;
where taselisib and palbociclib have the structures: 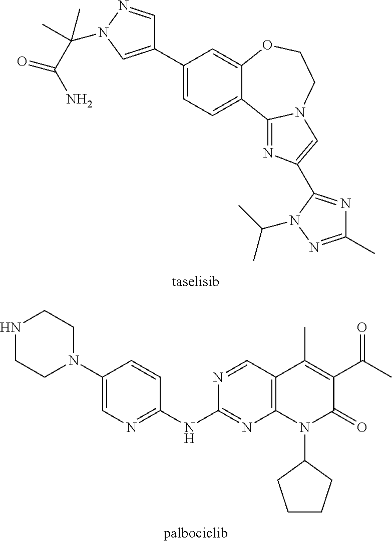 [SMILES: Cc1nc(-c2cn3c(n2)-c2ccc(-c4cnn(C(C)(C)C(N)=O)c4)cc2OCC3)n(C(C)C)n1 ; CC(=O)c1c(C)c2cnc(Nc3ccc(N4CCNCC4)cn3)nc2n(C2CCCC2)c1=O]
or stereoisomers, geometric isomers, tautomers, or pharmaceutically acceptable salts thereof.

2. The method of claim 1 wherein the therapeutically effective amounts of taselisib and palbociclib are administered as a combined formulation.

3. The method of claim 1 wherein the therapeutically effective amounts of taselisib and palbociclib are administered by alternation.

4. The method of claim 1 wherein the patient is administered with taselisib and subsequently administered with palbociclib.

5. The method of claim 1 wherein the therapeutic combination is administered by a dosing regimen where the therapeutically effective amount of taselisib is administered in a range from twice daily to once every three weeks, and the therapeutically effective amount of palbociclib is administered in a range from twice daily to once every three weeks.

6. The method of claim 5 wherein the dosing regimen is repeated one or more times.

7. The method of claim 1 wherein administration of the therapeutic combination results in a synergistic effect.

8. The method of claim 1 wherein the cancer is selected from breast, cervical, colon, endometrial, glioma, lung, melanoma, ovarian, pancreatic, and prostate.

9. The method of claim 8 wherein the cancer expresses a PIK3CA mutant selected from E542K, E545K, Q546R, H1047L and H1047R.

10. The method of claim 8 wherein the cancer expresses a K-ras mutant.

11. The method of claim 8 wherein the cancer expresses a PTEN mutant.

12. The method of claim 8 wherein the cancer is breast cancer.

13. The method of claim 12 wherein the breast cancer is HER2 positive.

14. The method of claim 12 wherein the breast cancer is HER2 negative, ER (estrogen receptor) negative, and PR (progesterone receptor) negative.

15. The method of claim 14 wherein the breast cancer is Basal subtype or Luminal subtype.

16. The method of claim 1 wherein taselisib and palbociclib are each administered in an amount from about 1 mg to about 1000 mg per unit dosage form.

17. The method of claim 1 wherein taselisib and palbociclib are administered in a ratio of about 1:50 to about 50:1 by weight.

18. The method of claim 1 wherein the cancer is a hormone-dependent cancer.

19. The method of claim 18 wherein the cancer is resistant to anti-hormonal treatment.

20. The method of claim 19, wherein the anti-hormonal treatment includes treatment with at least one agent selected from tamoxifen, fulvestrant, steroidal aromatase inhibitors, and non-steroidal aromatase inhibitors.

21. The method of claim 18 wherein the cancer is hormone receptor positive metastatic breast cancer.

22. The method of claim 21 wherein the therapeutic combination is administered to a postmenopausal woman with disease progression following anti-estrogen therapy.

23. The method of claim 1 wherein the pharmaceutically acceptable salt of taselisib or palbociclib is selected from a salt formed with hydrochloric acid, hydrobromic acid, hydroiodic acid, sulphuric acid, nitric acid, phosphoric acid, methanesulfonic acid, benzenesulphonic acid, formic acid, acetic acid, trifluoroacetic acid, propionic acid, oxalic acid, malonic acid, succinic acid, fumaric acid, maleic acid, lactic acid, malic acid, tartaric acid, citric acid, ethanesulfonic acid, aspartic acid and glutamic acid.

24. An article of manufacture for treating cancer comprising:
a) a therapeutic combination comprising a therapeutically effective amount of taselisib, and a therapeutically effective amount of palbociclib;
where taselisib and palbociclib have the structures: 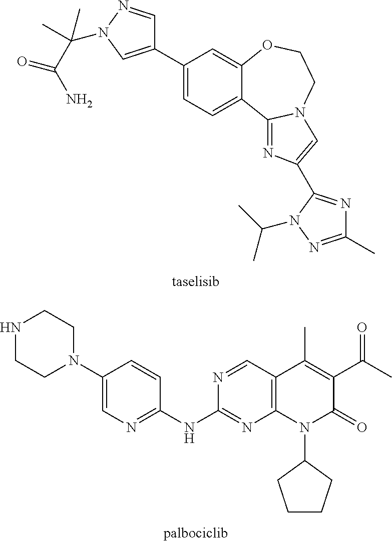 [SMILES: Cc1nc(-c2cn3c(n2)-c2ccc(-c4cnn(C(C)(C)C(N)=O)c4)cc2OCC3)n(C(C)C)n1 ; CC(=O)c1c(C)c2cnc(Nc3ccc(N4CCNCC4)cn3)nc2n(C2CCCC2)c1=O]
or stereoisomers, geometric isomers, tautomers, or pharmaceutically acceptable salts thereof; and
b) instructions for use.

25. The method of claim 1 wherein a biological sample obtained from the patient, prior to administration of the therapeutic combination to the patient, has been tested for PIK3CA or PTEN mutation status, and wherein PIK3CA or PTEN mutation status is indicative of therapeutic responsiveness by the patient to the therapeutic combination.

26. The method of claim 25 wherein the cancer is HER2 expressing breast cancer.

27. The method of claim 25 wherein the cancer is estrogen receptor positive (ER+) breast cancer.

28. The method of claim 25 wherein a biological sample has been tested by measuring functional PI3K protein level after administration of taselisib or the therapeutic combination, wherein a change in the level of functional PI3K protein indicates that the patient will be resistant or responsive to the therapeutic combination.

29. A method of monitoring whether a patient with cancer will respond to treatment with a therapeutic combination comprising a therapeutically effective amount of taselisib, and a therapeutically effective amount of palbociclib;
where taselisib and palbociclib have the structures: 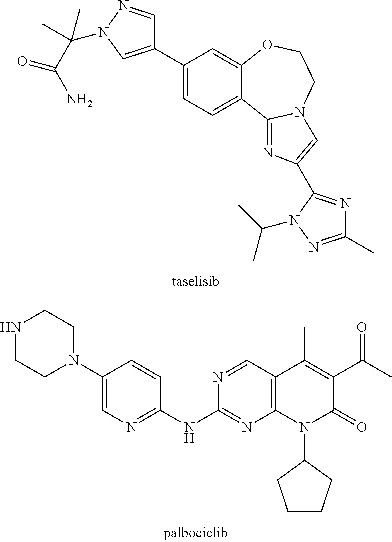 [SMILES: Cc1nc(-c2cn3c(n2)-c2ccc(-c4cnn(C(C)(C)C(N)=O)c4)cc2OCC3)n(C(C)C)n1 ; CC(=O)c1c(C)c2cnc(Nc3ccc(N4CCNCC4)cn3)nc2n(C2CCCC2)c1=O]
or stereoisomers, geometric isomers, tautomers, or pharmaceutically acceptable salts thereof;
the method comprising:
(a) detecting a PIK3CA or PTEN mutation in a biological sample obtained from the patient following administration of the at least one dose of taselisib or the therapeutic combination; and
(b) comparing PIK3CA or PTEN mutation status in a biological sample obtained from the patient prior to administration of taselisib or the therapeutic combination to the patient,
wherein a change or modulation of PIK3CA or PTEN mutation status in the sample obtained following administration of taselisib or the therapeutic combination identifies a patient who will respond to treatment with the therapeutic combination.

30. The method of claim 29 wherein the cancer is HER2 expressing breast cancer.

31. A method of optimizing therapeutic efficacy of a therapeutic combination comprising a therapeutically effective amount of taselisib, and a therapeutically effective amount of palbociclib;
where taselisib and palbociclib have the structures: 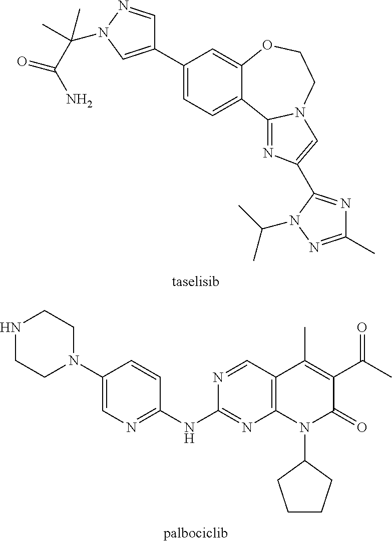 [SMILES: Cc1nc(-c2cn3c(n2)-c2ccc(-c4cnn(C(C)(C)C(N)=O)c4)cc2OCC3)n(C(C)C)n1 ; CC(=O)c1c(C)c2cnc(Nc3ccc(N4CCNCC4)cn3)nc2n(C2CCCC2)c1=O]
or stereoisomers, geometric isomers, tautomers, or pharmaceutically acceptable salts thereof;
the method comprising:
(a) detecting a PIK3CA or PTEN mutation in a biological sample obtained from a patient following administration of at least one dose of taselisib or the therapeutic combination; and
(b) comparing the PIK3CA or PTEN status in a biological sample obtained from the patient prior to administration of taselisib or the therapeutic combination to the patient,
wherein a change or modulation of PIK3CA or PTEN mutation status in the sample obtained following administration of taselisib or the therapeutic combination identifies a patient who has an increased likelihood of benefit from treatment with the therapeutic combination.

32. The method of claim 31 wherein the cancer is HER2 expressing breast cancer.

33. A method of identifying a biomarker for monitoring responsiveness a therapeutic combination comprising a therapeutically effective amount of taselisib, and a therapeutically effective amount of palbociclib;
where taselisib and palbociclib have the structures: 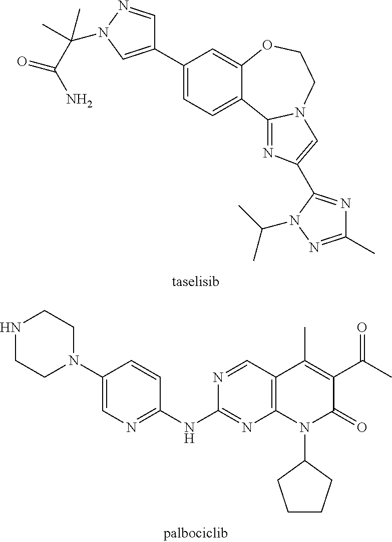 [SMILES: Cc1nc(-c2cn3c(n2)-c2ccc(-c4cnn(C(C)(C)C(N)=O)c4)cc2OCC3)n(C(C)C)n1 ; CC(=O)c1c(C)c2cnc(Nc3ccc(N4CCNCC4)cn3)nc2n(C2CCCC2)c1=O]
or stereoisomers, geometric isomers, tautomers, or pharmaceutically acceptable salts thereof;
the method comprising:
(a) detecting the expression, modulation, or activity of a biomarker mutation selected from a PIK3CA or PTEN mutation in a biological sample obtained from a patient who has received at least one dose of taselisib or the therapeutic combination; and
(b) comparing the expression, modulation, or activity of the biomarker mutation to the status of the biomarker in a reference sample wherein the reference sample is a biological sample obtained from the patient prior to administration of taselisib or the therapeutic combination to the patient;
wherein the modulation of the biomarker changes by at least 2 fold lower or higher compared to the reference sample is identified as a biomarker useful for monitoring responsiveness to the therapeutic combination.

34. The method of claim 33 wherein the cancer is HER2 expressing breast cancer.

35. The method of claim 33 wherein the biomarker mutation is the H1047R, H1047L, E542K, E545K or Q546R mutation of PIK3CA.

36. A use of a therapeutic combination comprising a therapeutically effective amount of taselisib, and a therapeutically effective amount of palbociclib;
where taselisib and palbociclib have the structures: 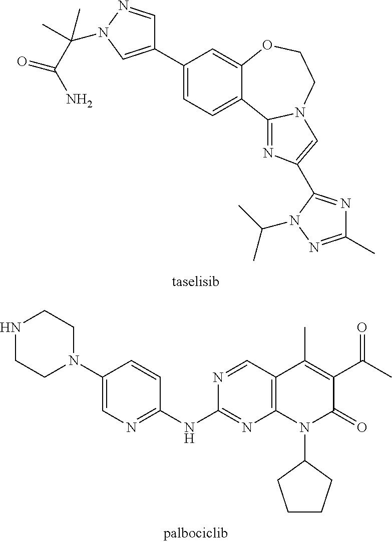 [SMILES: Cc1nc(-c2cn3c(n2)-c2ccc(-c4cnn(C(C)(C)C(N)=O)c4)cc2OCC3)n(C(C)C)n1 ; CC(=O)c1c(C)c2cnc(Nc3ccc(N4CCNCC4)cn3)nc2n(C2CCCC2)c1=O]
or stereoisomers, geometric isomers, tautomers, or pharmaceutically acceptable salts thereof;
in a patient comprising administering the therapeutic combination to a patient with cancer, wherein a biological sample obtained from the patient, prior to administration of the therapeutic combination, has been tested for PIK3CA or PTEN mutation status, and wherein PIK3CA or PTEN mutation status is indicative of therapeutic responsiveness by the patient to the therapeutic combination.

37. The use of claim 36 wherein the cancer is HER2 expressing breast cancer.

38. The use of claim 36 wherein the cancer is estrogen receptor positive (ER+) breast cancer.
